(19) United States
(12) Patent Application Publication
(10) Pub. No.: US 2017/0102378 A1
(43) Pub. Date: Apr. 13, 2017

(54) INDUSTRIAL DESIGN OF STAND-ALONE ASSAY SYSTEM

(71) Applicant: Silicon BioDevices, Inc., Palo Alto, CA (US)

(72) Inventors: Octavian FLORESCU, Berkeley, CA (US); Daniel WONG, Sunnyvale, CA (US); Igor IZYUMIN, Oakland, CA (US)

(73) Assignee: Silicon BioDevices, Inc., Palo Alto, CA (US)

(21) Appl. No.: 15/290,973

(22) Filed: Oct. 11, 2016

Related U.S. Application Data
(60) Provisional application No. 62/239,433, filed on Oct. 9, 2015.
(60) Provisional application No. 62/286,226, filed on Jan. 22, 2016.

(51) Int. Cl. G01N 33/53

(52) U.S. Cl. CPC G01N 33/5302

(57) ABSTRACT

A stand-alone assay system for POC IVD that is fast, easy to use, inexpensive and delivers laboratory quality results for one or more target analytes from a small volume of unprocessed aqueous sample is disclosed. The stand-alone assay system is a disposable digital device. The stand-alone assay system integrates a sampling system that relies on users swiping a lanced finger containing, an unprocessed aqueous sample such as whole blood across a sample inlet to transfer or deposit the unprocessed aqueous sample on to a membrane filter or into a capillary. The stand-alone assays system may have one or more wireless modules for wirelessly transmitting assay information. The wireless modules can receive or transmit user information like patient ID or assay information like time, location or calibration values. The stand-alone assay system can display and transmit the assay information simultaneously. The stand-alone assays system can transmit the assay information through more than one wireless modules simultaneously.

### CROSS-REFERENCE TO RELATED APPLICATIONS

The present application claims priority to U.S. Provisional Application Nos. 62/239,433, filed Oct. 9, 2015, and 62/286,226, filed Jan. 22, 2016, both of which are incorporated herein by reference in their entireties.

### BACKGROUND

1. Technical field

An assay system and method for use in the field of chemical testing is disclosed. More particularly, the assay system can be used for analyzing fluid samples quickly and easily with results wirelessly uploaded automatically for storage and analysis.

2. Summary of the related art

Point-of-Care (POC) diagnostic medical devices facilitate early stage detection of diseases, enable more individually tailored therapies, and allow doctors to follow up with patients more easily to see if prescribed treatments are working. To ensure widespread adoption, these tools must be accurate, easy to use by untrained individuals, and inexpensive to produce and distribute. In-vitro diagnostics (IVD) are particularly well-suited for the POC since a wide range of conditions, from cardiovascular disease to cancer to communicable infections, can be identified or monitored from soluble bio-markers. Glucose and pregnancy tests are two examples of POC IND platforms in widespread use. Both technologies provide valuable and high-quality results rapidly from an easy to obtain sample. Moreover, they are easy to use.

Emerging technologies, in particular micro-fluidics, have tried to replicate the success of these platforms but failed. The attempts that have met clinical performance guidelines have nonetheless been slow to be adopted by users, either professional or consumer. Often, these emerging technologies require multi-step procedures for activation cartridges must be taken out of the refrigerator and allowed to come up to room temperatures, the cartridge must be inserted into a read and discarded after use. The assay reader must be initialized and calibrated. Device lockout is problematic for higher volume settings. Moreover, the protocol for applying a sample is often multi-step involving additional transfer devices, diluents or complex sample preparation.

For POC application, it is desirable that the sample preparation be rapid since the assay is limited to 10-15 minutes. In addition, to obviate the need .for refrigeration and to facilitate storage and distribution, a dry sample preparation system is desirable. It is also desirable to have a sample preparation system that receives small unprocessed samples from patients. For example, the average hanging drop of blood from a finger stick yields approximately 15-20 μl of fluid. For more fluid, a complicated venu-puncture can be necessary. Moreover, the sample preparation system must be low-cost since biological contamination concerns dictate that all material in contact with biological samples be discarded. It is also desirable that the sample preparation system be amenable to multiplexed operation. Transfer devices and diluents add imprecision, increase the cost and complicate operation. Finally, all this functionality must be encapsulated in a system that costs no more than the marginal cost of a laboratory test.

Stand-alone operation is an often overlooked feature of successful POC EVD implementations. A stand-alone assay system is a device that does not require an external instrument or additional external components to function. An example is the home pregnancy test.

Stand-alone operation can he achieved through the use of magnetic particles as assay labels. The magnetic particles that react with one or more target analytes in the sample before sedimenting via gravity or magnetic force to a surface, where they can bind specifically and can be detected. A bio-functionalized IC can be used to detect the specifically bound particles.

Magnetic particle labeling is ideal for POC applications; magnetic particles can be individually detected; so sub-picomolar sensitivities can be achieved without signal amplification steps that can take up to an hour as in case of enzymatic labeling. Also, by micro-arraying the sensor areas onto which the particles bind, multiplexed operation can be achieved at low cost. The use of magnetic particles can reduce incubation times, since they can bind to the target analytes with solution-phase kinetics due to their high surface area to volume ratio. Furthermore, the ability to pull the magnetic particles out of solution magnetically and gravitationally overcomes the slow diffusion processes that plague most high sensitivity protocols. The signals from magnetic particles can be stable over time, insensitive to changes in temperature or chemistries and detected in opaque or translucent solutions like whole blood or plasma. The biological magnetic background signal can be low, so high assay sensitivity can be achieved with minimal sample preparation. Most importantly, the use of magnetic particles as assay labels can be both manipulated and detected electromagnetically, facilitating monolithic integration.

“Magnetic particles” are nanometer or micrometer sized particles that display magnetic, diamagnetic, ferromagnetic, ferrimagnetic, paramagnetic, super-paramagnetic or antiferromagnetic behavior, “Magnetic particles” can refer to individual particles or larger aggregates of particles such as magnetic beads.

Integrated circuits can monolithically integrate the magnetic particle detection and manipulation along with the necessary digital and analog signal processing thereby reducing system cost and providing; a simple user-experience.

Cost is an important consideration. The devices must be disposable after the first use for both bio-contamination and ease-of-use considerations.

The sampling technique employed must also be carefully designed. Transfer devices introduce user and analytical errors. Transfer pipettes are expensive and must work from a large sample volume while transfer capillaries are difficult to use and can introduce bubbles and other volumetric errors. The use of a transfer device makes the assay more difficult and costly to perform.

Finally, for stand-alone assay systems that can measure more than one target analytes, a robustly and easily wirelessly uploading results is useful. The telecommunication standard would be helpful to ensure that results are securely transmitted for storage and post-analysis.

### BRIEF SUMMARY OF THE INVENTION

A stand-alone assay system 1 for POC IVD that is fast, easy to use, inexpensive and delivers laboratory quality results for one or more target analytes 9 from a small volume of unprocessed aqueous sample 2 is disclosed.

The stand-alone assay system 1 can be a disposable digital device. The stand-alone assay system 1 can integrate a sampling system that relies on users swiping a lanced finger containing an unprocessed aqueous sample 2 such as whole blood across a sample inlet 5 to transfer or deposit the unprocessed aqueous sample 2 on to a membrane filter 11 or into a capillary 10. The internal sample 8 can be the filtrate of the unprocessed aqueous sample 2. The internal sample 8 precisely fill a metered assay chamber 7 containing magnetic particles 13 stored in a dry state. The magnetic particles 13 can be re-hydrated by the internal sample 8 and sediment to the bio-functionalized surface 16 of the IC 6. The IC 6 can generate magnetic forces to remove non-specifically bound particles from atop sensors 14 embedded in the IC 6. The bound magnetic. particles that remain specifically bound to the surface 16 of the IC 6 can be detected or counted by the sensors 14 embedded in the IC 6 and the count can be correlated to a concentration of one or more target analytes 9.

The stand-alone assay system 1 can have one or more buttons 40 for activation, one or more batteries 17 for power and a digital display 3 for displaying results. The stand-alone assays system 1 may have one or more wireless modules for wirelessly transmitting assay information 19. The wireless modules can receive or transmit user transmitting information like patient ID or assay information like time, location or calibration values.

The stand-alone assay system 1 can display and transmit the assay information 19 simultaneously. The stand-alone assays system 1 can transmit the assay information 19 through more than one wireless modules simultaneously.

A stand-alone assay system is disclosed that can have an IC, an assay chamber above the IC, a membrane filter, a capillary below the membrane filter, a top surface with an inlet above the membrane filter, a battery, an assay result display, or combinations thereof. The IC can be horizontal. The assay chamber can contain a precise volume of filtrate. The membrane filter can be used to filter whole blood delivered to the filter. The capillary can wick the filtrate from the bottom of the membrane filter into the assay chamber. The battery can deliver power to at least the IC. The display can have a digital display.

The inlet can be configured to collect whole blood from a sample surface swiped by a lanced (e.g., bleeding or bloody) finger across the top surface. The whole blood in the inlet can be wicked into the membrane filter. The IC can measure a target analyte in the filtrate in the assay chamber.

The inlet can have a pronounced edge and a flat edge. The pronounced edge can be above or higher than the flat edge. The pronounced edge can be above the bottom of the inlet. The pronounced or hard edge can be above the bottom of the inlet. The flat edge can be flush with the bottom of the inlet.

The top surface can be hydrophobic or non-stick.

The system can have one or more wireless modules.

The IC can have electrodes. The electrodes can perform electro-chemistries, for example on the filtered blood or analytes.

The inlet can block the membrane filter from contact by a user during use, for example so users cannot touch the filter.

The system can have a physical mesh between the filter and the inlet. The mesh can protect the filter from contact by a user during use (e.g., swiping of a finger).

The flat edge can be configured to not collect a sample off a swiped surface. The pronounced edge can be configured to collect a sample off a swiped surface. For example, the pronounced edge can be above the flat edge. Furthermore, exemplary radii of the edges are disclosed. This can, for example, be a squeegee or a lip that the user can run their lanced finger on to drop the blood into the inlet.

A method of using a stand-alone assay system is disclosed. The method can include lancing a body part. Lancing can allow a body fluid to exit the body part. The method can also include swiping the body part over a squeegee or top surface on the assay system. The squeegee can direct the body fluid into an inlet on the assay system. The assay system can include an IC, an assay chamber above the IC, a membrane filter, a capillary below the membrane filter. The method can include filtering the body fluid through the membrane filter, wicking the filtrate from the bottom of the membrane filter into the assay chamber, measuring a target analyte in the filtrate in the assay chamber with the and combinations thereof.

The body part can be a finger. The body fluid can be or include whole and/or pre-filtered blood.

### BRIEF DESCRIPTION OF THE DRAWINGS

FIG. 1 is a perspective view of the stand-alone assay system 1 with in sample inlet 5, a digital display 4 and a top surface 4

FIG. 2 is a cross sectional drawing of the sample inlet 5 with a sharp or pronounced edge 30 and a flat edge 34

FIG. 3 is an exploded view of the stand-alone assay system 1 having an assay sub-assembly 49 and a battery.

FIG. 4 is a picture of a user pressing button 46 on the side of the stand-alone assay system.

FIG. 5 is a cross-sectional diagram of the assay-subassembly 49 having a membrane filter 11, a capillary 10, an assay chamber 7, magnetic particles 13 and an IC 6.

### DETAILED DESCRIPTION

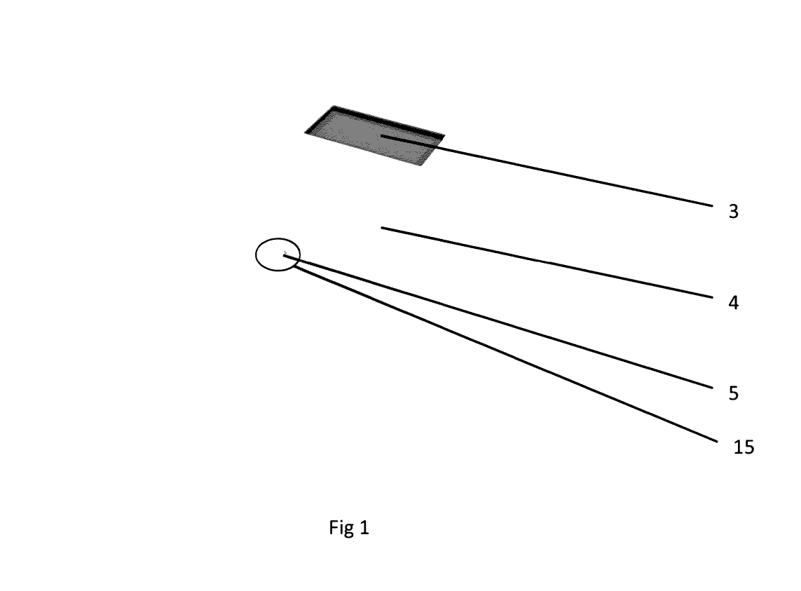

FIG. 1 is a perspective view of the stand-alone assay system 1. The stand-alone assay system 1 can contain a digital display 3 and a sample inlet 5 for collecting an unprocessed aqueous sample 2 and a top surface 4. The digital display 3 can be placed on the top surface 4 of the stand-alone assay system 1. The sample inlet 5 can be an aperture in the top surface 4 of the stand-alone assay system 1. The length of the digital display 3 can be oriented along the width of the top surface 4. The length of the digital display 3 can be oriented along the length of the top surface 4. The top surface 4 can be square, rectangular, trapezoidal or any other shape with rounded or had edges. Users can apply an unprocessed aqueous sample 2 to the sample inlet 5. The sample inlet 5 can be positioned so that the unprocessed aqueous sample 2 can flow on to membrane filter 11. The membrane filter 11 can be in proximity to the sample inlet 5 so that the unprocessed aqueous sample can be sipped or wicked from the sample inlet 5. The membrane filter 11 can be fitted above a. capillary 10 that wicks the internal sample 8 from the bottom of the membrane filter 11 into assay chamber 7. Alternatively, the unprocessed aqueous sample 2 can be directly wicked from the sample inlet 5 by the capillary 10 into the assay chamber without passing through a membrane filter.

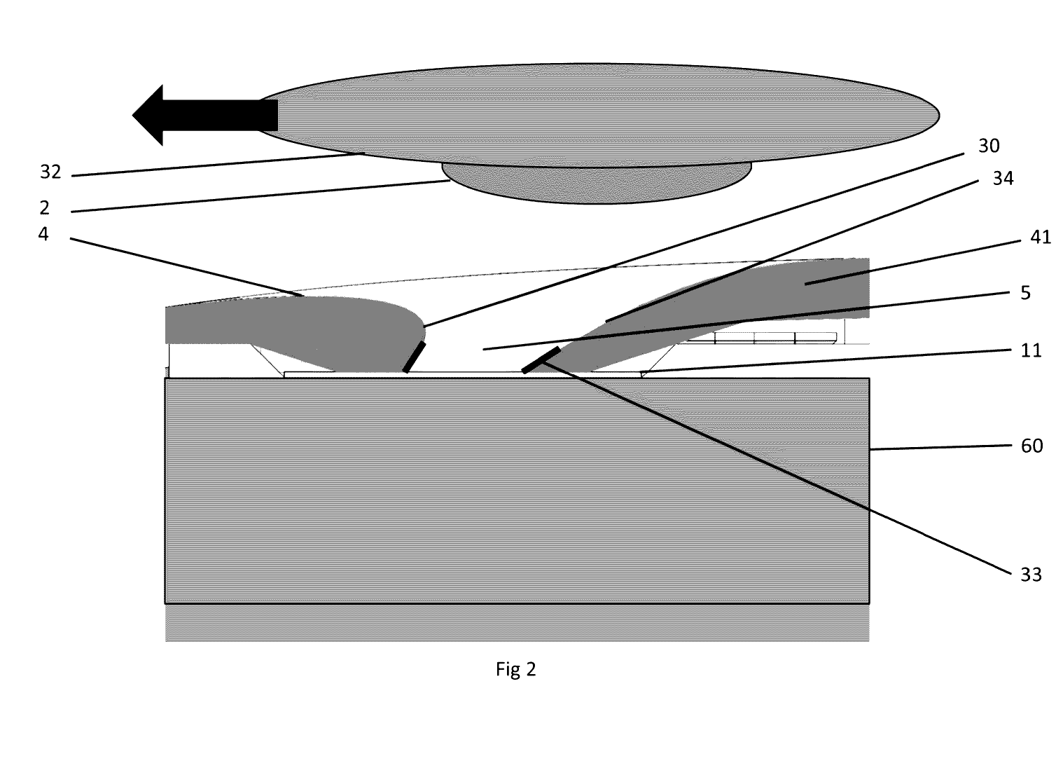

FIG. 2 is a cross sectional drawing of the sample inlet 5. The sample inlet 5 can have a sharp or a more pronounced edge 30 to collect the unprocessed aqueous sample 2 and a flat edge 34 to facilitate swiping of a sample surface 32 containing an unprocessed aqueous sample. The sample surface 32 can be above the sample inlet 5. Users can swipe a sample surface 32 containing an unprocessed aqueous sample from the flat edge 34 towards the pronounced edge 30 to run, transfer, drop or squeegee the unprocessed aqueous sample 2 from the sample surface 32 into sample inlet 5. The sample surface 32 can be a finger, a body part or other surface. A lancet finger stick can result in blood running across a finger. By running, transferring, dropping, swiping or squeegee-ing the sample surface 32 in contact with the unprocessed aqueous sample 2 against the sharp or pronounced edge 30, the unprocessed aqueous sample can be concentrated, accumulated or deposited by the sharp or pronounced edge 30 into the sample inlet 5. The top surface 4 of the stand-alone assay system 1 can be hydrophobic or non-stick to ensure that little to no unprocessed aqueous sample 2 is lost by adhesion to the top surface 4. Less than 0.1 μL, 0.5 μL, 1 μL, 5 μL, 10 μL of the unprocessed aqueous sample 2 can be lost by adhesion to the top surface when a sample surface 32 is swiped across sample inlet 5. The top surface 4 can be made non-stick or hydrophobic by dipping into a solution, spraying or vapor coating. The sample inlet 5 can have an inner surface 55 which can be the concentric surface in the lower portion or at the bottom of the sample inlet 5. The entirety or part of the inner surface 33 of the sample inlet 5 can be rendered non-stick or hydrophobic. The sharp or pronounced edge 30 can be adjacent, nearby or overlapping the sample inlet 5. The Sharp or pronounced edge 30 can define a contour or a portion of the sample inlet 5.

Users can lance a finger or other body part and swipe the lanced location over the sample inlet 5. Users can milk or press a finger or other body part multiple times once or repeatedly. Users can repeatedly swipe the sharp or pronounced edge 30 of the sample inlet 5 to deposit more unprocessed aqueous sample 2.

The stand-alone assay system 1 can accept less than 1 μl, 5 μl, 10 μl, 15 μl, 20 μl, 25 μl, 30 μl, 35 μl or more than 35 μl of unprocessed aqueous sample 2. The assay system may work properly when overfilled. The sample inlet 5 may have a fill line 15 along the perimeter to inform users how much unprocessed aqueous sample 2 is required. The fill line 15 can be inside the sample inlet 5, along the perimeter of the sample inlet 5 or outside the sample inlet 5. The sipping of the aqueous sample 2 by the membrane filter 11 may take more than 1 second, 10 seconds, 30 seconds, 1 minute.

The inner surface 33 of the sample inlet 5 can be hydrophilic or sticky to ensure that unprocessed aqueous sample 2 sticks to the inner surface 33. The sample inlet 5 can be an aperture to a capillary 10 that sips or wicks the unprocessed aqueous sample 2. The sample inlet 5 can be above, can touch or can press the membrane filter 11. The sample inlet 5 can be placed on the side of the stand-alone assay system 1 to sip from as sample surface from the side.

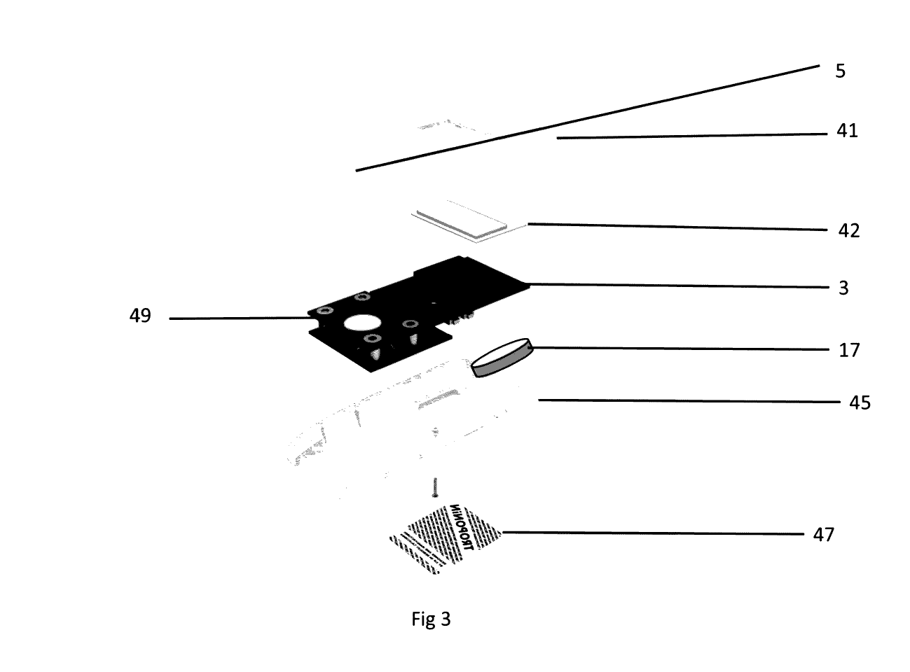

FIG. 3 shows an exploded view of the stand-alone assay system 1. The stand-alone assay system 1 can contain one or more batteries 17, a digital display 3, a sample inlet 5, a button 46 and a product label 47 printed with the molecule of interest, the device serial number and other information. The stand-alone assay system 1 can have the assay sub-assembly 49, a lens 42, a top cover 41 and a bottom cover 45. The top cover 41 and the bottom cover 45 can be injection molded plastic pieces. The top cover 41 and bottom cover 45 can be treated with surfactants and sterilized.

The top cover 41 can have a top surface 4, and sample inlet 5 and an opening for a display lens 42 which can be flush with the top surface 4. Flushness can be important to avoid the unprocessed aqueous sample 2 leaving residue on the top surface 4 due to ridges and irregularities. The bottom cover 45 can have living hinges for the button 46 and for a hinged door to release the battery 17 once the stand-alone assay system 1 is to be discarded

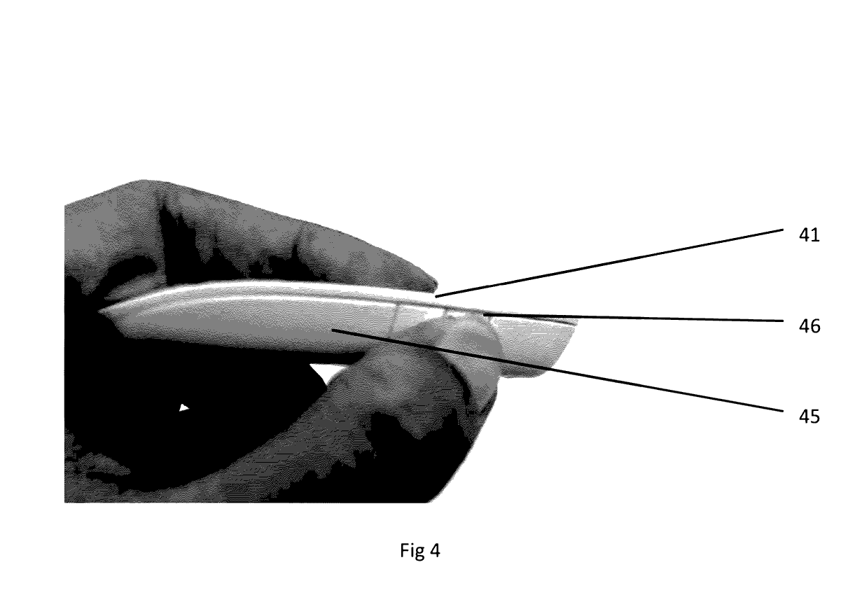

FIG. 4 shows a user pressing the button 46. The button 46 can be a separate piece of injection molded plastic or combined with the top cover 41 or bottom cover 45 using a living hinge. The stand-alone assay system 1 can contain more than one button. Users can press the button 46 to activate the assay, control the assay procedure, or wake up the digital display 3 once the assay is complete.

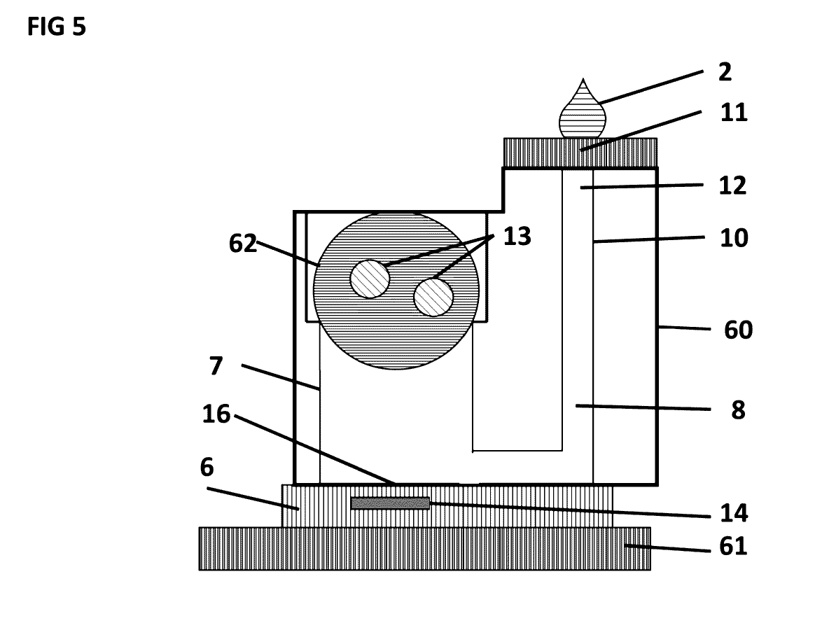

FIG. 5 is a cross sectional view showing the assay sub-system 49. The assay sub-system 49 can have an integrated circuit (IC) 6 mated to one or more assay chambers 7, file assay chamber 7 can be manufactured with precise tolerances and can passively fill with a metered amount of internal sample 8. The volume of the assay chamber 7 can have 0.1%, 1%, 2%, 3%, 4%, 5%, 6%, 7%, 8%, 9%, 10% or higher imprecision. The IC 6 can measure the amount target analyte 9 in the metered internal sample 8 in the assay chamber 7. The IC 6 can be mounted in a Printer Circuit Board (PCB) 61. The PCB 61 can have electrical connections to the digital display 3 and to the one or more batteries 17.

The stand-alone assay system 1 can perform immunoassays, small molecule assays, general chemistry measurements, blood gas measurements, cell counts, nucleic acid detection, nucleic add sequencing or other analytical measurements sequentially or concurrently in one or more adjacent assay chambers. The stand-alone assay system 1 can perform sandwich immuno-assays, or competitive immuno-assays.

The stand-alone assay system 1 can be sterile. Any external surface of the stand-alone assay system 1 that can be touched can be sterile. The top surface and the filter can be sterile. Any portion of the assay system can be sterilized chemically, with heat. or with various forms of radiation such as ultraviolet or acoustic.

Unprocessed aqueous samples 2 can be processed to yield the internal sample 8. The unprocessed aqueous sample 2 can flow directly via one or more capillaries 10 to the assay chamber 7 to yield the internal sample 8. The internal sample 8 is the fluid inside the assay chamber 7. The internal sample 8 can be filtered unprocessed aqueous sample 2. The internal sample 8 can be unprocessed aqueous sample 8 in which other chemicals are added or removed. The unprocessed aqueous sample 2 can be whole blood, serum, plasma, urine, tear, sputum, fecal, oral, nasal samples or other biological or non-biological aqueous samples.

A porous material like a membrane filter 11 can he fitted to top of the fluidic assembly 60. The fluidic assembly 60 can contain the capillary 10, the assay chamber 7 and the magnetic particles 13. The membrane filter 11 can process the unprocessed aqueous sample 2 and obviate the need for centrifugation or complicated microfluidic sample preparation. Since membrane filters are compact and inexpensive, system cost is reduced, enabling stand-alone POC operation. Furthermore, the membrane filters 11 can separate the plasma from the whole blood cells without additional user intervention or equipment in under 30 seconds. The membrane filter 11 can be placed horizontally, above the inlet 12 of one or more capillaries 10 that is arranged to allow the internal sample 8 to flow into and precisely fill one or more assay chambers 7.

The IC 6 can be placed horizontally with one or more exposed sensors 14 embedded under the surface 16 of the IC 6, below one or more assay chambers 7. Bio-chemically functionalized magnetic panicles 13 and other reagents can be stored in a dried state in the membrane filter 11, the capillary 10, the sensor areas on the surface of the IC, or combinations thereof The magnetic particles 11 can be dried, and stored at the inlet 12 of the capillary, on the surface 16 of the EC 6 inside the assay chamber 7, or above assay chamber 7. An unprocessed aqueous sample 2, such as whole blood, containing one or more target analytes 9 can be deposited in the sample inlet 5 above or near the membrane filter 11. The large particulate matter in the sample, such as whole blood cells, can be trapped on top or in the membrane filter 11, while the internal sample 8 containing the target analytes 9 flows into the inlet 12 of the capillary 10 and into the assay chamber 7. The magnetic particles 13 can be re-hydrated by the internal sample 7 and can react with the target analytes 9 in the internal sample 7. The re-hydrated magnetic particles 13 can sediment onto the on the surface 16 of the IC 6. The dried, magnetic particles 13 can stored in a dry sphere 62.

Magnetic particles 13 that react to one or more target analytes 9 in the internal sample 8 can bind strongly through specific chemical interactions to the functionalized the surface 16 of the IC 6. The number of magnetic particles 13 specifically bound to the surface 16 of the IC 6 is representative of the concentration of the target analyte 9 in the unprocessed aqueous sample 2 presented onto the membrane filter 10.

The IC 6 can contain one or more on-chip magnetic force generators to concentrate sedimenting magnetic particles and separation specifically bound magnetic particles from unbound ones. Magnetic concentration force generators can be configured to attract magnetic particles 13 above sensors 14. Magnetic separation force generators can be configured to remove non-specifically bound magnetic particles from above the sensors 14. The dried magnetic particles 13 can be placed at the top of the assay chamber 7. The rehydrated dried magnetic particles 13 can sediment to the surface 16 of the IC 6, above the sensors 14. The length of time of the assay can be determined by the height of the assay chamber 7. The sensors 14 can be magnetic particles sensors that can detect one or more magnetic particles 13.

Chemicals, such as, but not limited to: aptamers, oligonucloetides, proteins, agents to prevent clotting, target analytes for internal calibration curves, bindive catalytic agents, magnetic particles, or combinations thereof may be dried in the membrane filter 11 along the capillary 15, on the surface 16 of the IC 6 or in the dried reagents at the top of the assay chamber 7, and can be re-solubilized by the internal sample 8. These chemical can be dissolved into the internal sample 8 or remain bound to the surface upon which they were dried.

The stand-alone assay system 1 can provide results within 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes or more from application of the unprocessed aqueous sample 2.

In an immuno-assay format, the magnetic particles 13 can react to the target analyte 9 by binding through an antigen-antibody bond. In a nucleic acid assay format, the magnetic particles 13 can react to the target analyte 9 by binding through a complementary oligo-nucleotide bond.

The stand-alone assay system 1 can contain a battery 17 fur power and a digital display 3 to display assay information 19. The IC 6 can contain all the analog and digital signal processing functions to control the digital display 3. The PCB 61 can integrate analog and digital signal processing functions to control the digital display.

The system 1 can have multiple unconnected capillaries 10 below multiple unconnected membranes to deliver distinct internal samples 8 to multiple unconnected assay chambers 7 above one or more ICs 6 for multiplexed operation.

The system 1 can have a membrane filter 11 or one or more membrane filter assemblies comprising multiple stacked membrane filters 11 of the same or varying characteristics, where each filter can be loaded with different dried proteins, reagents, chemicals, magnetic particles and combinations thereof.

The characteristics of the membrane filter 11 can be varied to accommodate the different sample types. The membrane filter 11 can be replaced or used in conjunction with a porous material like a glass fiber or a nitrocellulose strip.

The stand-alone assay system 1 can be activated when a user presses button 46. Upon activation, a first rendering on the digital display 3 can be a logo such as a company logo. The first rendering can transition to a second or subsequent rendering automatically after a delay. The first rendering on the digital display can be omitted and once the assay system is turned on the digital display can display the second or subsequent rendering.

The second or subsequent rendering on the digital display 3 can display the name of the molecule of interest. The second or subsequent rendering on the digital display 3 can display an indication to deposit an unprocessed aqueous sample 2. The indication to deposit an unprocessed aqueous sample 2 can contain the words “swipe finger” or “deposit” or “place” or “drop” or “drip” or “put” or “add” or “touch” or “collect” or “pipette” or “milk” or “apply” and “press”. The indication to deposit an unprocessed aqueous sample can also be an icon of a drop, or an icon of a drop over an inlet or surface.

The user can be prompted to press a button 46 a second time once the unprocessed aqueous sample 2 is applied into the sample inlet 5. Once the user presses the one or more button 46 a second time the third or subsequent rendering can appear an the digital display 3 and the assay can begin. The third or subsequent rendering can display the name of the molecule of interest. The third or subsequent rendering can display an icon or indication to wait. The third or subsequent rendering can display the time until the analysis is complete. The digital display can display the time until the assay is complete in seconds, minutes and hours, through an hourglass or with a progress or status bar. The time until the assay is complete can refresh every second or every second, 2 seconds, or every 5 seconds, or every 10 seconds or every 20 seconds or every 30 seconds or every minute. The third or subsequent rendering can display a status bar that can refresh every second or every 2 seconds, or every 5 seconds, or every 10 seconds or every 20 seconds or every 30 seconds or every minute.

Once the assay is complete the results can be displayed in the fourth or subsequent rendering. The fourth or subsequent rendering can display a qualitative or quantitative measurement of the concentration of the target analyte 9 in the unprocessed aqueous sample 1. The fourth or subsequent rendering can display assay information 19. The assay information 19 can include the assay results, the time, the date, the assay system serial number, the patient ID or other information. The fourth or subsequent rendering can display a 1-Dimensional or 2-Dimensional code. The quantitative or qualitative measurement of the concentration of the target analyte 9 can be encoded or encrypted in the 1-dimensional or 2-dimensional code. The time, the date, the assay system serial number or other assay information can be displayed in the 1-dimensional or 2-Dimensional code. A secondary mobile device can be used to machine-read the 1-dimensional or 2-Dimensional code to retrieve the assay information. The digital display 3 can be an LCD, LED OLED or other digital display. Each element of the 1 dimensional or 2-Dimensional code can be rendered by one pixel of the display, or 4 pixels of the display, or 9 pixels of the display, or 16 pixels of the display.

The first rendering, the second rendering, the third rendering and the fourth rendering can appear in any order or can be omitted.

Only once the assay information 19 is uploaded to a secondary mobile device, the stand-alone assay device 1 can be deactivated using the one or more buttons 46. The battery 17 of the stand-alone assay system 1 can be removed by using a finger or other apparatus to open a hinged door on the bottom cover 45 of the stand-alone assay system 1. The hinge door can break away along an intentionally weakened segment or it can contain a living hinge. Users can open the hinge and gain access to the one or more batteries. The battery 17 can be removed from the stand-alone assay system 1 and discarded separately or retained.

The stand-alone assay system 1 may contain one, two, three, four or more separate wireless modules. One example of a wireless module is an optical wireless module using the digital display 3. An optical reader such as a secondary mobile device with a camera can be used to machine reach the digital display 3. The secondary mobile device can have software to de-crypt and translate the assay information on digital display 3.

The wireless module can have a nearfield communication module (NEC)—e.g. ISO/IEC 14443. NFC can be an attractive wireless module due to its low cost, security, and ease of integration. The assay information 19 must be associated with a patient ID from whom the sample 2 was taken. This may be done by either interrogating the stand-alone assay system 1 with an NEC reader (such as a smartphone), or by sequentially scanning a serial number or barcode identifying the stand-alone assay system 1 and a patient's user ID, barcode or serial number. The identifiers or the patient and the stand-alone system can be uploaded in any order. In a hospital setting, many stand-alone assay systems 1 may be in use simultaneously for different patients, and it is desirable to automatically transmit the assay results to the medical records system with no further staff intervention once an assay is complete. This can be accomplished by placing the stand-alone assay system on an NEC reader as soon as the assay is started, before the assay is started, before the unprocessed aqueous sample is placed on the stand-alone assay system 1, or after the sample is placed on the assay systems 1. Such a NFC reader may be in the shape of a mat or a surface on which one or more stand-alone assay systems 1 may be placed while an assay is running, or contain a flat or almost flat surface on which to place the stand-alone assay system. The NFC reader may poll stand-alone assay systems 1 placed on its surface to monitor the progress of each assay. Once the assay is complete, or while the assay is in progress, the NFC reader may transmit the results to the medical records system, and optionally provide an audiovisual indication that a test is finished. Automatic upload of test results minimizes staff time required to run an assay, and avoids error-prone manual steps. An audiovisual, alarm signal may he provided to warn staff that the stand-alone system 1 was placed on the NFC reader mat, or has not been associated with a patient (e.g. if the staff member neglected to scan the assay system barcode). An audiovisual alarm signal may also indicate a result that needs to be addressed immediately.

A thin protective sheet of paper or plastic or other material can be included in the stand-alone system 1 packaging to place between the re-usable NEC reader and the disposable assay system 1 that has hazardous samples placed on it. The thin protective sheet can be discarded with the assay system once the assay is complete.

Another embodiment of the NFC reader can be attached to a biohazardous waste receptacle. Used stand-alone assay systems 1 are placed in a tray or slot on the NFC reader, where they are interrogated by an NFC coil. An audiovisual indication can be provided if the assay results were read successfully. At that point, the user may actuate a button or lever on the NFC reader causing the stand-alone assay system 1 to enter the biohazardous waste receptacle. In another embodiment, this mechanism may be actuated automatically via a motor or solenoid once stand-alone assay system 1 is interrogated successfully. In another embodiment, the NFC reader includes one or more slots or trays that allow the stand-alone assay system to rest in an appropriate orientation (e.g. horizontal) to permit the assay to run. Once the assay is complete or otherwise terminated and the assay system is successfully interrogated by the NFC reader, an actuator opens, flips, or rotates the tray or slot door to cause the assay system to frill into the waste receptacle. This embodiment has multiple advantages in a clinical setting: minimal bench space is required, staff contact with bodily fluids is minimized, results are uploaded automatically with minimal assay system handling, and used stand-alone assay systems are separated from other waste.

The communication protocol may incorporate encryption and authentication protocols to ensure patient data security and regulatory compliance. In one embodiment, a stand-alone assay system 1 may use a cryptographic certificate to verify that the NFC reader is authorized to access assay data. Likewise, the NFC reader may use a cryptographic certificate to verify that a stand-alone assay system 1 is genuine. Appropriate encryption techniques should be used to ensure regulatory compliance (e.g. HIPAA, FIPS 140-2, and relevant NIST standards).

All functions performed by an NFC reader can also be implemented by an optical reader that can machine-read the digital display 3 or other optical wireless module in the stand-alone assay system 1.

To ensure that the assay system is placed on the NFC reader, or on a flat stable surface, the assay system 1 may visually request through the digital display 3, once the sample 2 has been applied, that the assay system 1 be placed on the NFC reader. Once assay information 19 is available, it can be uploaded directly through the NFC reader into medical databases. The assay system 1 can detect proximity to the NFC reader to ensure that it is placed flat during operation. If close proximity to the NFC reader is not detected, an error message can be displayed or communicated through a wireless modules. An NFC antenna can be integrated on the PCB 61.

Upon activation, the stand-alone assay system 1 can display a message indicating the amount of time allotted before sample 2 must be introduced. During this sample introduction time, no buttons 46 may work and the digital display may not change until the sample is introduced. if no sample 2 is introduced, at the end of the sample introduction time, the stand-alone assay system 1 will display a sample introduction error as the result. After the sample analysis is complete, assay information 19 will be displayed in an encoded or up-encoded format. Assay information 19 can remain displayed on the digital display 3 more than 1 minute, 2 minutes, 10 minutes, 30 minutes, 1 hour, 2 hours, 1 day, 2 days, 1 week, 1 month, 1 year or until the batter discharges. Alternatively, assay information 19 can be displayed for a predetermined result display time, which can be 10 seconds, 30 seconds, 1 minute, 2 minutes, 5 minutes, 10 minutes, 30 minutes, after which time the assay system 1 goes into a results standby mode where the digital display is turned oft to conserve battery charge. In standby mode, a user can press a button 46 to re-display the assay information 19 for the result display time. Alternatively, to obviate the need for a button 46, the assay information 19 can be displayed with a duty cycle. To conserve batter y charge the assay information 19 can be displayed for a results display time after which the digital display is automatically turned, off for a standby time. At the end of the standby time, the cycle restarts. The digital display can be flashed on and off to convey that the test has been completed.

At any point, the assay system 1 can flash the display to convey urgency, completion, attention is needed, action is needed or errors.

The assay system 1 can display a number of statuses not limited to on/off, requesting a sample, analyzing result, time remaining, the results and errors. Errors can be but are not limited to internal self-testing errors, sample introduction time violation, tilting of the assay system, invalid controls, or others.

The assay system 1 can have one or more patient identifiers or identification elements, for example the patient ID and the assay system ID presented on the box, the foil pouch, the assay system 1 or the digital display 3. The assay system ID and the patient 110 can be bound together in medical databases for billing and data handling. Alternatively, the assay system ID can be read by a secondary mobile device that can also be used to input the patient ID through the keyboard, fingerprint sensor or camera. A photo of the patient can also be included in the patient ID.

The standalone assay system 1 may contain multiple wireless modules. The assay system can have a port for wired communication. The assay system can have an NFC module for near field communication. The assay system can also transmit optically through the digital display or other embedded optical wireless module& The assay system can communicate through multiple wireless modules simultaneously. The wireless modules can be integrated on the PCB 61, the IC 6, or the digital display 3.

The present application incorporates by reference in their entirety U.S. Pat. Nos. 8,895,320, filed May 14, 2012 and issued November 25, 2014, 9,244,068, filed Oct. 24, 2014 and issued Jan. 26, 2016; and U.S. patent application Ser. Nos. 14/878,760, filed Oct. 8, 2015, 13/858,794, tiled Apr. 8, 2013, and 14/942,903, filed Nov. 16, 2015. Any details and elements in the aforementioned patents and patent applications can he used in conjunction and combination with the disclosure herein.

Variations of the systems, devices and methods have been shown and described herein by ways of example only. Variations, changes, and substitutions can occur. For example, the methods can be performed with any one or more elements of the methods absent, and any one or more element of the devices can be omitted. Various alternatives and combinations of elements between the variations described herein may be employed. All publications, patents, and patent applications mentioned in this specification are herein incorporated by reference to the same extent as if each individual publication, patent, or patent application was specifically and individually indicated to be incorporated by reference.

We claim:

1. A stand-alone assay system comprising:
an IC placed horizontally;
an assay chamber above the IC containing a precise volume of filtrate;
a membrane filter to filter whole blood;
a capillary below the membrane filter that is configured to wick the filtrate from the bottom of the membrane filter into the assay chamber;
a top surface with an inlet above the membrane filter;
a battery, wherein the system is configured so the battery at least delivers power to the IC; and
an assay result display, wherein the display comprises a digital display;
wherein the inlet, is configured to collect whole blood from a sample surface swiped across the top surface, and wherein the whole blood in the inlet is wicked into the membrane filter, and wherein the IC measures a target analyte in the filtrate in the assay chamber.

2. The system of claim 1, wherein the inlet has a pronounced edge and a flat edge, and wherein the pronounced edge is above the flat edge.

3. The system of claim 1, wherein the top surface can be hydrophobic or non-stick.

4. The system of claim 1, further comprising a wireless module.

5. The system of claim 1, wherein the IC comprises electrodes.

6. The system of claim 5, wherein the electrodes are configured to perform electro-chemistries.

7. The system of claim 2, wherein the pronounced edge is above the bottom of the inlet.

8. The system of claim 2, wherein the pronounced edge can be above the bottom of the inlet.

9. The system of claim 8, wherein the flat edge is flush with the bottom of the inlet.

10. The system of claim 2, wherein the flat edge is flush with the bottom of the inlet.

11. The system of claim 1, wherein the inlet blocks the membrane filter from contact by a user during use.

12. The system of claim 1, further comprising a physical mesh between the filter and the inlet, wherein the mesh is configured to protect the filter from contact.

13. The system of claim 1, wherein the flat edge is configured to not collect a sample off a swiped surface, and wherein the pronounced edge is configured to collect a sample off a swiped surface.

14. A method of using a stand-alone assay system comprising:
lancing a body part, wherein the lancing comprises allowing a body fluid to exit the body part;
swiping the body part over a squeegee on the assay system, wherein the squeegee directs the body fluid into an inlet on the assay system, and wherein the assay system further comprises an IC, an assay chamber above the IC, a membrane filter, a capillary below the membrane filter
filtering the body fluid through the membrane filter;
wicking the filtrate from the bottom of the membrane filter into the assay chamber; and
measuring a target analyte in the filtrate in the assay chamber with the IC.

15. The method of claim 14, wherein the body part comprises a finger.

16. The method of claim 14, wherein the body fluid comprises blood.
